(19) United States
(12) Patent Application Publication
(10) Pub. No.: US 2025/0302327 A1
(43) Pub. Date: Oct. 2, 2025

(54) MEDICAL SYSTEMS, DEVICES, AND METHODS FOR IMPEDANCE-GUIDED TISSUE SAMPLE COLLECTION

(71) Applicant: Boston Scientific Scimed, Inc., Maple Grove, MN (US)

(72) Inventors: Ajay GUPTA, Minneapolis, MN (US); Celeste GIDEON, Kansas City, KS (US); Andrew CZARNIAK, Plymouth, MN (US); Kialie MALONE, Minneapolis, MN (US); Grace MORGAN, Mankato, MN (US); Nicole VU, Shoreview, MN (US); Isabella KELLER, Noblesville, IN (US); Brooke BENNETT, Hackettstown, NJ (US); Olivia Dominique KALATA, LaGrange, IL (US)

(73) Assignee: Boston Scientific Scimed, Inc., Maple Grove, MN (US)

(21) Appl. No.: 19/086,297

(22) Filed: Mar. 21, 2025

Related U.S. Application Data
(60) Provisional application No. 63/569,851, filed on Mar. 26, 2024.

(51) Int. Cl. A61B 5/0537; A61B 5/00; A61B 10/02

(52) U.S. Cl. CPC A61B 5/0537; A61B 5/7264; A61B 5/742; A61B 10/0233

(57) ABSTRACT

A medical device includes an end effector assembly at a distal end of the medical device. The assembly comprises a pair of jaw members, a pair of electrodes including first and second electrodes, and first and second conductive wires attached to the first and second electrodes, respectively. Each jaw member includes a jaw and a tang, the jaw being transitionable between open and closed configurations for respectively receiving and collecting a tissue portion as a sample. The first and second conductive wires extend to a proximal end of the medical device and are removably connectable to a computing device configured to generate and provide electrical signals to, and determine an impedance measurement of the tissue portion based on response signals received from, the pair of electrodes contacting the tissue portion, and generate and provide a user interface indicating whether to collect the tissue portion based on the impedance measurement.

### CROSS-REFERENCE TO RELATED APPLICATIONS

This application claims the benefit of priority to U.S. Provisional Application No. 63/569,851, filed on Mar. 26, 2024, which is incorporated by reference herein in its entirety.

### TECHNICAL FIELD

The disclosure relates generally to medical systems, devices, and methods for tissue sample collection. More specifically, aspects of the disclosure pertain to medical systems, devices, and methods implementing in situ impedance analysis to guide a tissue sample collection determination.

### BACKGROUND

A medical system used to perform a biopsy procedure may include a sample collection device, such as biopsy forceps, that is delivered to a target site via a working channel of an endoscope or other similar medical imaging device. The sample collection device may be used to collect portions of tissue at the target site as samples for diagnostic analysis. Beyond inferences made based on images of the portions of tissue captured by the medical imaging device, an operator of the medical system may have limited knowledge as to whether the portions of tissue that are being collected are in fact areas of interest (e.g., suspect areas) for the diagnostic analysis.

### SUMMARY

According to one aspect, the techniques described herein relate to medical devices. An example medical device includes an end effector assembly at a distal end of the medical device, the end effector assembly including: a pair of jaw members, each of the pair of jaw members including a jaw and a tang, the jaw being transitionable between an open configuration for receiving a portion of tissue and a closed configuration for collecting the portion of tissue as a sample; and a pair of electrodes including a first electrode and a second electrode; a first conductive wire attached to the first electrode; and a second conductive wire attached to the second electrode, wherein each of the first conductive wire and the second conductive wire extend to a proximal end of the medical device and are removably connectable to a computing device configured to generate and provide electrical signals to the pair of electrodes as the pair of electrodes are contacting the portion of the tissue, determine an impedance measurement of the portion of the tissue based on response signals received from the pair of electrodes, and generate and provide a user interface indicating whether to collect the portion of tissue as the sample based on the impedance measurement.

In some aspects, each of the pair of electrodes is a portion of a body of the jaw of one of the pair of jaw members. The jaw of each of the pair of jaw members is further transitionable to an intermediate configuration for the impedance measurement, the intermediate configuration positioned between the open configuration and the closed configuration. The medical device further includes an actuator assembly at the proximal end of the medical device, where the actuator assembly includes a stop that maintains the jaw of each of the pair of jaw members in the intermediate configuration. In the intermediate configuration, a contact surface area between the pair of electrodes and the portion of the tissue is maintained, and a pressure of a particular value is applied to the portion of the tissue.

In other aspects, the pair of electrodes are separate from the pair of jaw members. Additionally, the pair of electrodes may be independently actionable from the pair of jaw members. For example, in response to an actuation of an actuator assembly, the pair of electrodes transition from an open configuration to a closed configuration for the impedance measurement as the pair of jaws remain in the open configuration. In the closed configuration of the pair of electrodes, a contact surface area between the pair of electrodes and the portion of the tissue is maintained, and a pressure of a particular value is applied to the portion of the tissue.

In further aspects, the medical device includes an instrument having the pair of electrodes disposed on a distal end of the instrument. The instrument is transitionable between a retracted configuration and an extended configuration relative to the end effector assembly. In some examples, the instrument is a needle, and in response to an actuation of an actuator assembly, the distal end of the instrument transitions to the extended configuration such that the distal end extends distally from the end effector assembly and at least partially into the portion of tissue for the impedance measurement as the pair of jaws remain in the open configuration.

In some aspects, the medical device includes a member connecting the end effector assembly to an actuator assembly at the proximal end of the medical device. In some examples, the first conductive wire and the second conductive wire extend within the member to the proximal end of the medical device. Additionally, the medical device may further include a control wire attached to the actuator assembly and to the end effector assembly, and extending within the member, where the first conductive wire and the second conductive wire extend within the control wire to the proximal end of the medical device. In other examples, the first conductive wire and the second conductive wire extend along an exterior of the member to the proximal end of the medical device.

According to another aspect, the techniques described herein relate to medical systems. An example medical system includes a sample collection device and a computing device. The sample collection device including: a pair of jaw members, each of the pair of jaw members including a jaw and a tang, the jaw being transitionable between an open configuration for receiving a portion of tissue and a closed configuration for collecting the portion of tissue as a sample; a pair of electrodes including a first electrode and a second electrode; a first conductive wire attached to the first electrode; and a second conductive wire attached to the second electrode. The computing device is removably and electrically connected to the pair of electrodes via the first conductive wire and the second conductive wire. The computing device includes at least one memory configured to store instructions, and at least one processor configured to execute the instructions to perform operations including: generating and providing electrical signals to the pair of electrodes; determining an impedance measurement for the portion of tissue based on response signals received as the pair of electrodes are contacting the portion of tissue; and based on the impedance measurement, generating and providing a user interface for display, the user interface indicating whether to collect the portion of tissue as a sample.

In some aspects, the portion of tissue is a second portion of tissue associated with suspect tissue, the response signals are second response signals, and the operations further include: determining a baseline impedance measurement based on first response signals received as the pair of electrodes are contacting a first portion of tissue associated with non-suspect tissue; determining a difference between the baseline impedance measurement and the impedance measurement; and comparing the difference to a predetermined threshold, wherein the user interface is generated further based on the comparing.

In other aspects, generating the user interface includes one of: generating a first user interface to indicate to collect the second portion of tissue as the sample when the difference meets or exceeds the predetermined threshold; or generating a second user interface to indicate not to collect the second portion of tissue as the sample when the difference fails to meet or exceed the predetermined threshold.

In further aspects, the operations further include determining a type of the tissue based on the response signals, where the user interface further displays the type of the tissue.

According to further aspects, the techniques described herein relate to methods. An example method includes: performing a first actuation to move a control wire, connected to an end effector assembly, in a first direction to cause a conductive pair of jaws of the end effector assembly having received a portion of tissue therein to transition from an open configuration to an intermediate configuration, wherein a computing device to which the conductive pair of jaws are connected to provides electrical signals to, and determines an impedance measurement of the portion of tissue based on response signals received from the conductive pair of jaws that are contacting the portion of the tissue in the intermediate configuration; and based on the impedance measurement, performing a second actuation to further move the control wire in the first direction to cause the conductive pair of jaws to transition from the intermediate configuration to a closed configuration to collect the portion of the tissue as a sample.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 depicts an example environment for impedance-guided tissue sample collection.

FIG. 2A depicts a partial cross-sectional view of an example first sample collection device.

FIG. 2B depicts a distal portion of the first sample collection device delivered to a target site via an example medical device.

FIGS. 3A and 3B depict a cross-section of a first configuration of the distal portion of the first sample collection device having open and closed jaw configurations, respectively.

FIGS. 4A and 4B depict a cross-section of a second configuration of the distal portion of the first sample collection device having open and closed jaw configurations, respectively.

FIGS. 5A and 5B depict a cross-section of a third configuration of the distal portion of the first sample collection device having open and closed jaw configurations, respectively.

FIG. 6 depicts the distal portion of the first sample collection device having an intermediate jaw configuration.

FIG. 7 depicts an example second sample collection device.

FIG. 8 depicts an example third sample collection device.

FIG. 9 depicts an example process for determining tissue impedance to guide a sample collection determination.

FIGS. 10A and 10B depict example user interfaces.

FIG. 11 depicts an example computing device.

### DETAILED DESCRIPTION

As briefly mentioned above, a medical system used to perform a biopsy procedure may include a sample collection device, such as biopsy forceps, that is delivered to a target site via a working channel of an endoscope or other similar medical imaging device. The sample collection device may be used to collect portions of tissue at the target site as samples for diagnostic analysis. Beyond inferences capable of being made based on images of the portions of tissue captured by the medical imaging device, an operator of the medical system has limited knowledge as to whether the portions of tissue that are being collected are in fact areas of interest (e.g., suspect areas) for the diagnostic analysis. Resultantly, a large number of negative samples that are not useful for the diagnostic analysis are often collected during the biopsy procedure.

Tissue impedance analysis may be performed to differentiate between different types of tissue. For example, impedance analysis may effectively differentiate between muscle tissue and fat tissue, between nerve tissue and other tissue types, and between malignant and benign tumor tissues, among other examples.

Therefore, aspects of this disclosure are directed to medical systems, devices, and methods for implementing in situ impedance analysis to guide a biopsy determination. For example, prior to collecting a portion of tissue as a sample, impedance of the tissue may be measured and analyzed to determine whether the portion of tissue is likely an area of interest for diagnostic analysis (e.g., is suspect or abnormal tissue). Results of the analysis may be output to the operator, and the operator may utilize this information to determine whether to collect the sample. Therefore, implementation of the impedance analysis may help to reduce a number of negative samples collected. Additionally, in at least some aspects, the impedance analysis may be performed on the portion of tissue without causing damage to the portion of tissue. Therefore, in instances, where the impedance analysis indicates the portion of tissue is not an area of interest, the portion of tissue remains undamaged, and the operator may simply move the sample collection device to a next portion of tissue to analyze for potential collection.

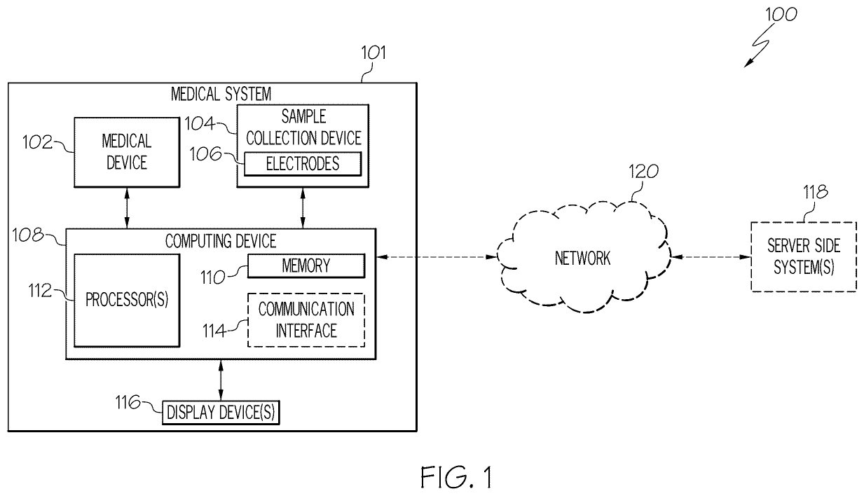

FIG. 1 depicts an exemplary environment 100 for impedance-guided sample collection. Environment 100 may include medical system 101, one or more optional server side system(s) 118, and/or an optional network 120 to communicatively couple one or more components of medical system 101 to optional server side system(s) 118. Medical system 101 may include one or more of a medical device 102, a sample collection device 104, a computing device 108, and one or more display device(s) 116.

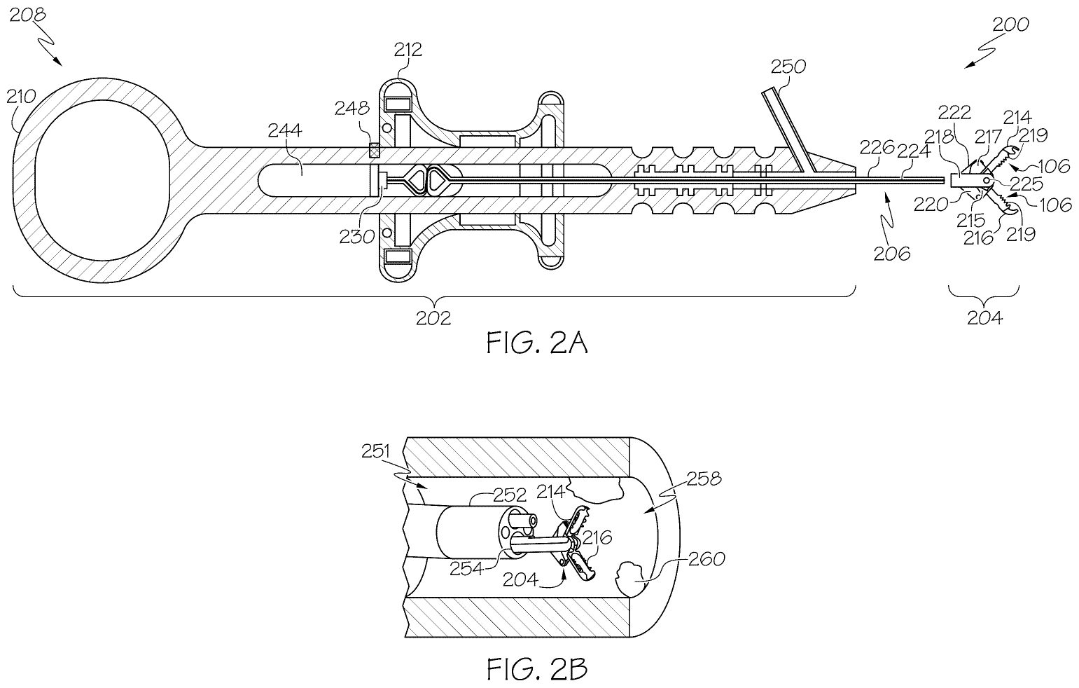

Medical device 102 may be used in conjunction with one or more other components of medical system 101 to perform a diagnostic medical procedure, such as a biopsy procedure, on a patient. For example, as part of the biopsy procedure, medical device 102 may be inserted into and navigated through a body lumen to identify, using images captured by medical device 102, a target site within the body lumen to collect tissue samples from. In some examples, and as described in more detail with reference to FIG. 2B, medical device 102 may be an endoscope or other type of scope (e.g., having imaging capabilities), such as a cholangioscope, bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples.

Sample collection device 104 may be an accessory device (e.g., a separate device, tool, or instrument from medical device 102) that is used in conjunction with medical device 102 to perform at least one or more operations of the biopsy procedure. For example, once medical device 102 has been navigated to the target site, at least a distal portion of sample collection device 104 may be delivered to the target site via the medical device 102, as described in more detail with reference to FIG. 2B. Sample collection device 104 may include biopsy forceps, among other examples, having a distal end effector configured to collect a portion of tissue from the target site for diagnostic analysis by cutting, piercing, or otherwise separating the portion of tissue from the body lumen.

Additionally, sample collection device 104 may be further configured to obtain impedance measurements prior to the collection of the portion of tissue. For example, sample collection device 104 may include or be associated with a pair of electrodes 106. Electrodes 106 may be bipolar electrodes that are each integrated with sample collection device 104 in various configurations, as described in detail throughout the disclosure. Electrodes 106 may be connected via one or more wires and/or cables to computing device 108 to receive and transmit electrical signals to and from computing device 108 to facilitate the impedance analysis. Although examples of electrodes 106 shown and described throughout the disclosure include bipolar electrodes, in other examples, the pair of electrodes 106 may be monopolar electrodes, where one of electrodes 106 is integrated with sample collection device 104, and the other of electrodes 106 is positioned externally on skin of the patient, for example.

Computing device 108 may include a controller, a control unit, a computing device, an integrated circuit, or other similar standalone processing unit separate from and removably connectable to medical device 102 and/or sample collection device 104. In some examples, both of medical device 102 and sample collection device 104 may be removably connectable to computing device 108. In other examples, medical system 101 may include more than one computing device 108, where each of medical device 102 and sample collection device 104 may be removably connectable to a different computing device 108. Alternatively, computing device 108 may be integrated into one of medical device 102 and/or sample collection device 104.

Computing device 108 may include a memory 110 and one or more processor(s) 112. Memory 110 may store instructions to be executed by processor(s) 112 to cause computing device 108 to perform corresponding operations. Memory 110 may also include one or more data stores. Additionally or alternatively, computing device 108 may include one or more data stores separate from memory 110. In some examples, the processor(s) 112 may be or include a field-programmable gate array (FPGA), a digital signal processing (DSP) processor, a graphics processing unit (GPU), or the like.

At least a portion of the instructions stored in memory 110 and executable by at least one of processor(s) 112 of computing device 108 that is removably connectable to and/or integrated with medical device 102 may include one or more image processing operations, among other instructions. Additionally, processor(s) 112 may include at least one image processor configured to process, based on the stored instructions, image data captured by imaging components of medical device 102 and provided to computing device 108 to generate images.

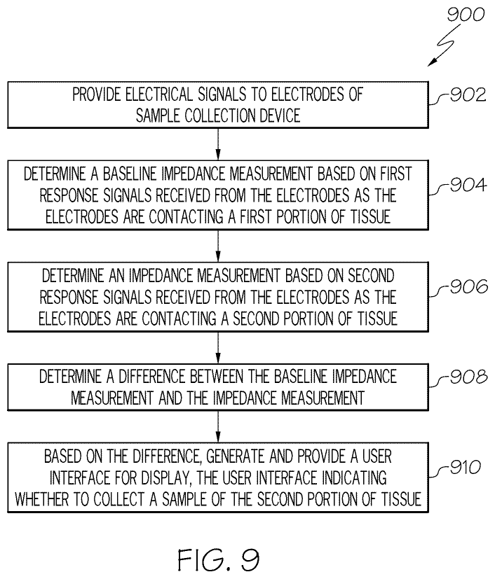

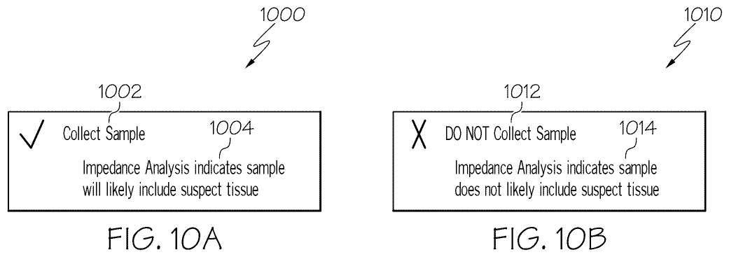

At least a portion of the instructions stored in memory 110 and executable by at least one of processor(s) 112 of computing device 108 that is removably connectable to and/or integrated with sample collection device 104 may include a process for determining and analyzing tissue impedance to guide a sample collection determination. As described in greater detail below with reference to FIG. 9, an example process may include: providing electrical signals to electrodes 106 of sample collection device 104; determining an impedance measurement based on response signals received as electrodes 106 are contacting a portion of tissue; and based on the impedance measurement, generating and providing a user interface for display (e.g., on one of display device(s) 116) that indicates whether to collect a sample of the portion of tissue. In some examples, one or more of the data stores included within or separate from memory 110 may store an impedance library for reference in the tissue impedance determination. In other examples, the impedance library may be stored remotely.

Computing device 108 may further include an optional communication interface 114 for providing connectivity to optional network 120 to facilitate communication with optional server side system(s) 118. Although not shown in FIG. 1, optional communication interface 114 may, in some examples, also provide connectivity to medical device 102, sample collection device 104, and/or display device(s) 116. In some examples, a communicative connection between computing device 108 and medical device 102, computing device 108 and sample collection device 104, and/or computing device 108 and display device(s) 116 may be at least partially supported via optional network 120.

Display device(s) 116 may be configured to display data associated with one or more of medical device 102, sample collection device 104, and/or computing device 108. In some examples, displayed data may include information associated with the tissue impedance determination and analysis process, including an indication of whether a portion of tissue should be collected as a sample. Additionally, when medical device 102 includes the imaging capabilities, the displayed data may also include images generated by computing device 108. Display device(s) 116 may include one or more a combination of monitors, computing device screens, touch screen display devices, etc. In some examples, one or more of display device(s) 116 may be a separate device from computing device 108 that is communicatively coupleable to computing device 108 via wired and/or wireless connections. In other examples, at least one of display device(s) 116 may be a display or screen that is integrated into the at least one of computing device 108.

In some examples, computing device 108 may generate, or may cause to be generated, one or more graphical user interfaces based on instructions or information stored in memory 110, instructions or information received from one or more optional server side system(s) 118, and/or the like and may cause the graphical user interfaces to be displayed via display device(s) 116. The graphical user interfaces may include text, visual elements, controls, and/or the like, in addition to the displayed data. Display device(s) 116 may include a touch screen or a display with other input systems (e.g., a mouse, keyboard, voice, etc.) for an operator of computing device 108 to control functions of computing device 108, medical device 102 or sample collection device 104 via computing device 108, and/or display device(s) 116.

One or more components of environment 100, such as medical device 102, sample collection device 104, computing device 108, and/or display device(s) 116, may be capable of network connectivity, and may communicate with one another over a wired network or a wireless network, such as optional network 120. The network may be an electronic network. The network may include a wide area network (“WAN”), a local area network (“LAN”), personal area network (“PAN”), a cellular network (e.g., a 3G network, a 4G network, a 5G network, etc.), or the like. In other examples, the components of environment 100 may communicate and/or connect to the network over universal serial bus (USB) or other similar local, low latency connections or direct wireless protocol. Components of environment 100 may be connected via the network, using one or more standard communication protocols, such that the component may transmit and receive communications from each other across the network.

In some examples, when one or more of the components of environment 100, such as computing device 108, are capable of connecting to optional network 120, environment 100 may also include optional server side system(s) 118. Optional server side system(s) 118 may include one or more remote data storage systems for storing data generated by computing device 108 (e.g., data associated with the tissue impedance determination process, including the impedance library, and/or image data). Additionally or alternatively, when medical device 102 includes an imaging system or device, optional server side system(s) 118 may include remote image processing systems configured to perform at least a portion of the image processing, including but not limited, more resource intensive processes, such as machine learning processes (e.g., to conserve local resources of computing device 108 when network connectivity is available).

Although various components in environment 100 are depicted as separate components in FIG. 1, it should be understood that a component or portion of a component in environment 100 may, in some embodiments, be integrated with or incorporated into one or more other components. For example, one of display device(s) 116 may be integrated with one of computing device 108 (s). In some embodiments, operations or aspects of one or more of the components discussed above may be distributed amongst one or more other components. Any suitable arrangement and/or integration of the various systems and devices of environment 100 may be used.

The specific examples included throughout the present disclosure describe an endoscopic biopsy system utilizing biopsy forceps that are configured to obtain impedance measurements of a portion of tissue prior to collecting the portion of tissue as a sample to facilitate a determination of whether or not the sample should in fact be collected. However, it should be understood that techniques according to this disclosure may be adapted to other medical systems having similar accessory devices for collecting tissue samples. For example, the techniques may be adapted to endoscopic biopsy systems utilizing core biopsy needles. It should also be understood that the examples above are illustrative only. The techniques and technologies of this disclosure may be adapted to any suitable activity.

FIG. 2A depicts a partial cross-sectional view of an example first sample collection device 200, hereinafter device 200 for brevity. Device 200 may be one example type of sample collection device 104 of medical system 101 described above with reference to FIG. 1. Device 200 includes an actuator assembly 202 (of which a cross-sectional view is shown in FIG. 2A) at a proximal end of device 200, an end effector assembly 204 at a distal end of device 200, and an elongate member 206 that connects actuator assembly 202 to end effector assembly 204.

Actuator assembly 202 may include a handle 208 with a thumb ring 210 and a spool 212. Spool 212 is movable along handle 208 between a proximalmost position and distalmost position of spool 212 on handle 208. Spool 212 is sized and shaped to be grasped by an operator of device 200. A distance between the proximalmost position and distalmost position may be a first distance.

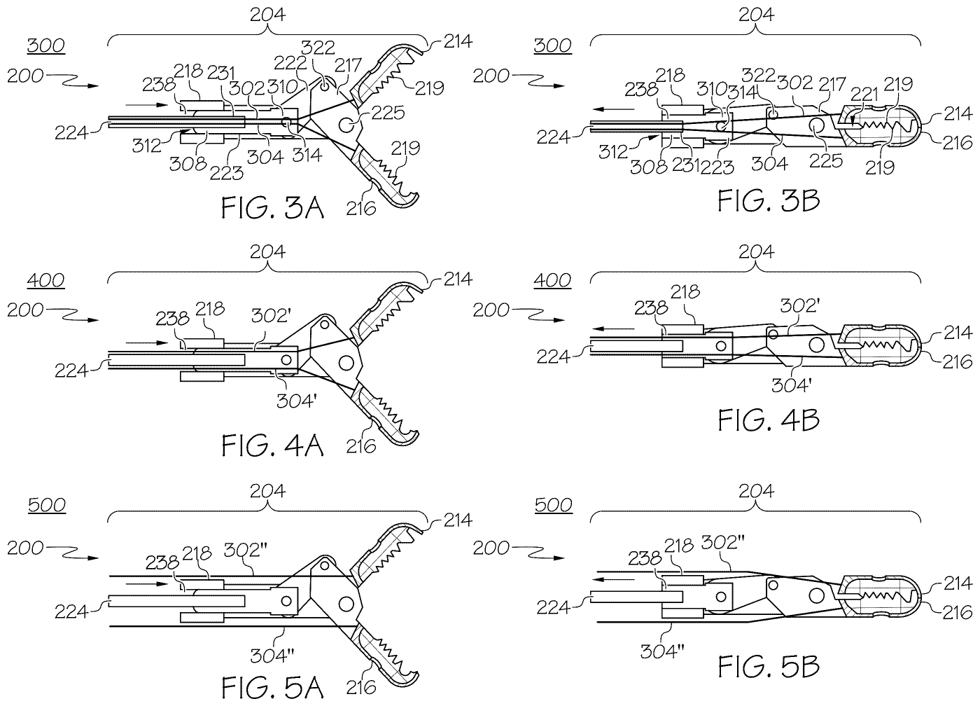

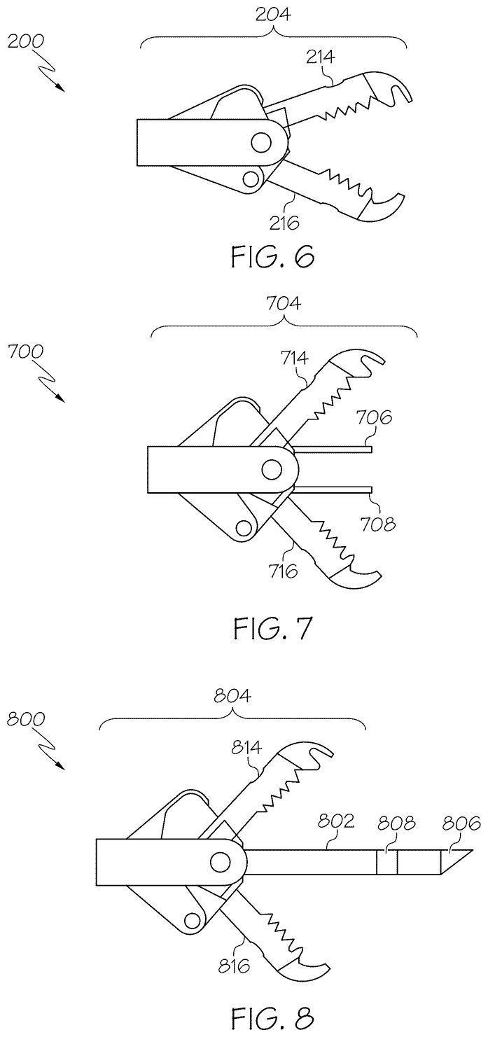

End effector assembly 204 may include first and second jaws 214, 216 (e.g., a pair of jaws), a clevis 218, first and second links 220, 222 and a control wire attachment 223 (FIGS. 3A-B). As described in more detail below, control wire attachment 223 may be a body that couples a control wire 224 to end effector assembly 204. First and second jaws 214, 216 may be transitionable between at least an open configuration and a closed configuration. In some examples, and as described in more detail with reference to FIG. 6, first and second jaws 214, 216 may be further transitionable to an intermediate configuration positioned between the open and closed configurations.

First and second jaws 214, 216 may include a generally cup-shaped body with convex outer surfaces and concave inner surfaces that, in the closed configuration, define an inner tissue-receiving space 221 (FIG. 3B) between first and second jaws 214, 216. Outer perimeter edges of the body of first and second jaws 214, 216 may be formed as tissue cutting edges 219 configured to mate with one another when in the closed configuration. In some examples, tissue cutting edges 219 may include serrations along a length of tissue cutting edges 219, the serrations having teeth. Tissue cutting edges 219 of first and second jaws 214, 216 may be complimentary such that peaks of teeth forming serrations of first jaw 214 fit within valleys of teeth forming serrations of second jaw 216 and vice versa. This offset fit of the teeth of first and second jaws 214, 216 may provide a clean cut on a portion of tissue to be collected without damaging either the tissue or first and second jaws 214, 216.

First and second jaws 214, 216 may also include first and second tangs 215, 217. First and second tangs 215, 217 may extend proximally from the body of first and second jaws 214, 216, respectively. Each of first and second tangs 215, 217 may include a pivot hole that is sized and shaped to receive a pivot pin 225 therethrough. Pivot pin 225 may be configured to extend through the pivot hole of each of first and second tangs 215, 217 transverse to a central longitudinal axis, L, of device 200. Additionally, an actuating pin 322 (FIGS. 3A-B) may extend from an outer surface of each of first and second tangs 215, 217 to pivotably connect first and second tangs 215, 217 to first and second links 220, 222, respectively. Each of links 220, 222 may also be pivotably connected to control wire attachment 223 via a link pin 314 (FIGS. 3A-3B), as discussed in further detail below.

In device 200, at least a portion of the body of each of first and second jaws 214, 216 may be conductive, and configured to serve as electrodes 106. In other words, bodies of jaws 214, 216 may themselves be electrodes 106. For example, at least the portion of the body of first and second jaws 214, 216 may be composed of a non-neutral, non-inert metal material capable of: (1) applying electrical signals through a portion of tissue received by first and second jaws 214, 216, and (2) receiving response signals that may be quantified to determine an impedance of the portion of tissue. The electrical and response signals may be respectively transmitted and received while first and second jaws 214, 216 are in at least a partially open configuration such that first and second jaws 214, 216 are contacting the portion of tissue but are not yet cutting or severing the portion of tissue.

Electrodes 106 integrated within the body of first and second jaws 214, 216 may be electrically isolated from one another. For example, one or more components of first and second jaws 214 separating the electrode portions thereof, such as first and second tangs 215, 217 extending from bodies of first and second jaws 214, 216, may be comprised of insulative material. Conductive wires and/or cables, such as first and second conductive wires 302, 304 (FIGS. 3A-B, 4A-B, and 5A-5B), may extend proximally from the electrode portions of the body of first and second jaws 214, 216 toward handle 208 to removably and electrically connect the electrode portions to computing device 108. As described in detail below, each of FIGS. 3A-B, FIGS. 4A-B, and FIGS. 5A-5B depict different configurations in which first and second conductive wires 302, 304 may be integrated with components of device 200.

Computing device 108 may generate and transmit electrical signals to and receive response signals from the electrode portions of the body of first and/or second jaws 214, 216 via first and/or second conductive wires 302, 304. As described in detail with reference to FIG. 9, computing device 108 may process the response signals to determine an impedance measurement of the portion of tissue received by first and second jaws 214, 216, and based on the impedance measurement, determine whether or not to recommend the portion of tissue be collected as a sample.

Clevis 218 may have a pair of arms (e.g., may be substantially U-shaped) and include a central lumen 238 to receive control wire attachment 223 (FIGS. 3A-B). A space defined between the arms of clevis 218 may be configured to receive first and second tangs 215, 217 of first and second jaws 214, 216, where each arm may be pivotably connected to first and second tangs 215, 217 via pivot pin 225. Control wire attachment 223 extends through central lumen 238 of clevis 218 and connects to a distal end 231 of control wire 224 (FIGS. 3A-B).

Elongate member 206 may be a coiled member, and houses a tension member (e.g., control wire 224) that extends from actuator assembly 202 to end effector assembly 204. For example, elongate member 206 may include a coil 226. Elongate member 206 may extend proximally from clevis 218 to connect to handle 208. Handle 208 may be configured to be receive coil 226. Coil 226 may be a cable configured to house one or more wires, such as control wire 224 that connects to control wire attachment 223 (FIGS. 3A-B) and extends proximally within, and is configured to slide relative to, elongate member 206.

A proximal end 230 of control wire 224 couples to spool 212. Spool 212 may include an interior surface that slides along the outside of handle 208 and a slot 244. Thus, movement of spool 212 relative to the handle 208 moves control wire 224 within coil 226 to permit actuation of end effector assembly 204. For example, distal movement of spool 212 relative to handle 208 causes control wire 224 to move distally, and first and second jaws 214, 216 to transition from a closed configuration to an open configuration to, for example, receive a portion of tissue to be collected as a sample. A subsequent proximal movement of spool 212 relative to handle 208 causes control wire 224 to move proximally, and first and second jaws 214, 216 to transition from the open configuration to a closed configuration to cut or sever the portion of tissue from the body lumen to collect a sample. As previously mentioned, an impedance measurement may be obtained and analyzed prior to fully transitioning first and second jaws 214, 216 from the open configuration to the closed configuration to confirm the portion of tissue is in fact an area of interest for diagnostics (e.g., is suspect tissue), and thus the sample should be collected.

In some examples, handle 208 may include a structural element, such as a stop 248, configured to facilitate separate actuation operations as spool 212 is moved relative to handle 208 between the proximalmost position and the distalmost position of spool 212 on handle 208. For example, and as described in detail with reference to FIGS. 6 and 7, enabling separate actuation operations may help to obtain a more accurate impedance measurement of the portion of tissue prior to and independent of sample collection of the portion of tissue. In some examples, stop 248 may provide tactile or auditory feedback to an operator. While stop 248 is illustrated and described herein, other example structural elements may include a lock, a latch, a ratchet, etc. In other example configurations of device 200, stop 248 may be omitted.

In further examples, handle 208 may include a port 250. For example, in configurations of device 200 where the conductive wires and/or cables connecting the electrode portions of the body of first and second jaws 214, 216 to computing device 108 extend proximally within elongate member 206 toward handle 208, the conductive wires and/or cables may exit handle 208 via port 250 in order to removably connect to computing device 108. Port 250 may include a plug, socket, or other similar structure for coupling to an active cord. In other example configurations of device 200, port 250 may be omitted.

FIG. 2B depicts end effector assembly 204 delivered to a target site within a body lumen 258. For example, end effector assembly 204 may be delivered to the target site via an endoscope 251. Endoscope 251 may be one example of medical device 102 described above with reference to FIG. 1. Specifically, distal end 252 of endoscope 251 may be inserted into and navigated through body lumen 258 to reach the target site where tissue 260 is located. End effector assembly 204 may be inserted into and extended through and distally past a working channel of endoscope 251 via a distal opening 254 of the working channel to deliver end effector assembly 204 to the target site. During delivery, spool 212 may be at the proximalmost position of spool 212 on handle 208 such that first and second jaws 214, 216 are in the closed configuration. Upon delivery, spool 212 may be distally moved a first distance relative to handle 208 to the distalmost position of spool 212 on handle 208 to cause first and second jaws 214, 216 to transition to the open configuration to receive a portion of tissue 260 for impedance analysis and/or sample collection, as described in detail below.

FIGS. 3A and 3B depict a cross-section of a first configuration 300 of end effector assembly 204 of device 200 having first and second jaws 214, 216 in open and closed configurations, respectively. As previously mentioned above, clevis 218 includes central lumen 238 to receive control wire attachment 223 therein. Control wire attachment 223 extends from a proximal end to a distal end and includes a proximal portion 308 and a distal portion 310. Proximal portion 308 may be substantially cylindrical and defines a central opening 312 at the proximal end. Central opening 312 may be configured to receive and couple (e.g., via welding) to a distal end 231 of control wire 224. Distal portion 310 may include generally flat lateral surfaces, each lateral surface including link pin 314 extending laterally therefrom.

In the cross-section depicted in FIGS. 3A-B, second jaw 216 is shown pivotably connected to second link 222 via actuating pin 322 extending from second tang 217, where second link 222 is further pivotably connected to distal portion 310 of control wire attachment 223 via link pin 314. Resultantly, as control wire attachment 223 is moved proximally (e.g., via proximal movement of spool 212), second link 222 is pulled proximally and second tang 217 pivots relative to second link 222 about actuating pin 322 such that an angle between second jaw 216 and second link 222 changes from approximately 90 degrees, as shown in FIG. 3A, to approximately 180 degrees, as shown in FIG. 3B.

Although not shown in the cross-section depicted in FIGS. 3A-B, first jaw 214, via connections similar to the connections shown for second jaw 216, may be pivotably connected to first link 220 via actuating pin 322 extending from first tang 215, where first link 220 is further pivotably connected to distal portion 310 of control wire attachment 223 via link pin 314. Resultantly, as the control wire attachment 223 is moved proximally (e.g., via proximal movement of spool 212), first link 220 is also pulled proximally and first tang 215 pivots relative to first link 220 about actuating pin 322 such that an angle between first jaw 214 and first link 220 changes from approximately 90 degrees to 180 degrees. This motion pivots first and second jaws 214, 216 toward one another such that first and second jaws 214, 216 move from the open configuration shown in FIG. 3A to the closed configuration shown in FIG. 3B.

As described in detail with reference to FIG. 2A, in device 200, at least a portion of the body of each of first and second jaws 214, 216 may be conductive, and configured to serve as electrodes 106. First conductive wire 302 may removably and electrically connect first jaw 214 to computing device 108 and/or a source of power. Second conductive wire 304 may removably and electrically connect second jaw 216 to computing device 108 and/or a source of power.

In first configuration 300, control wire 224 may include a lumen. For example, control wire 224 may be a hypotube. First and second conductive wires 302, 304, which are coupled to first and second jaws 214, 216, may be bundled within or otherwise housed by/extend through the lumen of control wire 224. In some examples, first and second conductive wires 302, 304 may be bundled or housed within a separate cable disposed within and along the lumen of control wire 224. Control wire 224, and thus first and second conductive wires 302, 304 housed therein, may extend proximally through central lumen 238 of clevis 218 and elongate member 206 toward handle 208. In some examples, upon reaching handle 208, first and second conductive wires 302, 304 (e.g., individually or housed within the separate cable) may exit from control wire 224 and from handle 208 via port 250 (FIG. 2A) to removably connect to computing device 108. In other examples, control wire 224 itself may be conductive. In such examples, proximal ends of first and second conductive wires 302, 304 may connect to and extend from distal ends of control wire 224 to first and second jaws 214, 216. Additionally, a further conductive wire connected to control wire 224 at a proximal end of control wire 224 may extend and exit from handle 208 via port 250 (FIG. 2A) to removably connect to computing device 108.

FIGS. 4A and 4B depict a cross-section of a second configuration 400 of end effector assembly 204 of device 200 having first and second jaws 214, 216 in open and closed configurations, respectively. Second configuration 400 is the same as first configuration 300 described in detail with reference to FIGS. 3A-B, except for a positioning of first and second conductive wires 302′, 304′. For example, in second configuration 400, first conductive wire 302′ coupled to first jaw 214 and second conductive wire 304′ coupled to second jaw 216 extend proximally through central lumen 238 of clevis 218 and via elongate member 206 toward handle 208 along with but separately from control wire 224. In other words, in second configuration 400, first and second conductive wires 302′, 304′ are not disposed within a lumen of control wire 224. In some examples, upon reaching handle 208, first and second conductive wires 302′, 304′ may exit from handle 208 via port 250 (FIG. 2A) to removably connect to computing device 108.

FIGS. 5A and 5B depict a cross-section of a third configuration 500 of end effector assembly 204 of device 200 having first and second jaws 214, 216 in open and closed configurations, respectively. Third configuration 500 is the same as first configuration 300 described in detail with reference to FIGS. 3A-B, except for a positioning of first and second conductive wires 302″, 304″. For example, in third configuration 500, first conductive wire 302″ coupled to first jaw 214 and second conductive wire 304″ coupled to second jaw 216 each extend proximally along an exterior of clevis 218 and an exterior of elongate member 206 toward handle 208. In some examples, first and second conductive wires 302″, 304″ may extend into (e.g., are bundled or housed within) a lumen of a separate cable that extends proximally along the exterior of clevis 218 and elongate member 206 toward handle 208. Additionally, clevis 218 and/or elongate member 206 may include one or more structural features (e.g., fasteners) along exterior surfaces thereof to receive first and second conductive wires 302″, 304″ and/or the cable bundling first and second conductive wires 302″, 304″ such that at least portions of first and second conductive wires 302″, 304″ and/or the cable lay flush against the exterior surfaces to help prevent tangling.

FIG. 6 depicts an example of end effector assembly 204 of device 200 having first and second jaws 214, 216 positioned in an intermediate configuration. Any one of first configuration 300, second configuration 400, or third configuration 500 of device 200 described above with reference to FIGS. 3A-B, 4A-B, and 5A-B, respectively, may be configured to transition first and second jaws 214, 216 to the intermediate configuration of FIG. 6. The intermediate configuration may be positioned between the open configuration (e.g., shown in FIGS. 3A, 4A, 5A) and closed configuration (e.g., shown in FIGS. 3B, 4B, 5B) of first and second jaws 214, 216. The intermediate configuration may be associated with obtaining impedance measurements.

As described with reference to FIG. 2A, handle 208 of device 200 may include stop 248, or other similar structural element, configured to facilitate separate actuation operations as spool 212 is moved relative to handle 208. The separate actuation operations may cause the transitioning of first and second jaws 214, 216 between the open, intermediate, and closed configurations.

For example, and as described above with reference to FIG. 2B, first and second jaws 214, 216 may initially be in the closed configuration as end effector assembly 204 is delivered to the target site. When in the closed configuration, spool 212 may be in the proximalmost position of spool 212 on handle 208. A first actuation of actuator assembly 202 may be performed to transition first and second jaws 214, 216 from the closed configuration to the open configuration. The first actuation may include a distal movement of spool 212 a first distance from the proximalmost position to the distalmost position of spool 212 on handle 208, causing control wire 224 and control wire attachment 223 to move distally, and resulting in the open configuration of first and second jaws 214, 216. In some examples, as spool 212 is moved from the proximalmost position to the distalmost position, additional force may be applied by operator as spool 212 encounters stop 248 to bypass the intermediate configuration. A portion of tissue may be received within first and second jaws 214, 216 in the open configuration.

Then, a second actuation of actuator assembly 202 may be performed to transition first and second jaws 214, 216 from the open configuration to the intermediate configuration to obtain an impedance measurement of the portion of tissue. For example, the second actuation may include a proximal movement of spool 212 from the distalmost position until spool 212 encounters stop 248, causing control wire 224 and control wire attachment 223 to move proximally, and resulting in the intermediate configuration of first and second jaws 214, 216. In alternatives, stop 248 may be omitted and handle 208 may include one or more markings for indicating the intermediate configuration. A distance from the distalmost position of spool 212 on handle 208 to stop 248 may be a second distance that is less than the first distance between the proximalmost and distalmost positions of spool 212 on handle 208. In the intermediate configuration, first and second jaws 214, 216 may contact and apply pressure to, but do not yet pierce or cut, the portion of tissue. For example, a constant contact surface area between electrode portions of the body of first and second jaws 214, 216 and the portion of the tissue may be maintained, while a constant pressure is applied to the portion of the tissue. The contact surface area and/or an amount of pressure applied to the portion of tissue in the intermediate configuration may correspond to optimal value(s) for obtaining an impedance measurement. In some examples, the constant surface area and/or the amount of pressure applied to the portion of tissue in the intermediate configuration may be maintained for at least a duration of (e.g., is consistent throughout) the impedance reading for the portion of tissue to obtain the impedance measurement. Additionally, in further examples, the constant surface area and/or the amount of pressure applied and maintained in the intermediate configuration may be consistent across impedance readings for multiple portions of tissue.

If based on the impedance analysis, a determination is made that the portion of tissue is likely an area of interest and thus the portion of tissue should be collected as a sample, a third actuation of actuator assembly 202 may be performed to transition first and second jaws 214, 216 from the intermediate configuration to the closed configuration to collect the sample. For example, the third actuation may include an application of force to overcome or cause a release of stop 248, and a further proximal movement of spool 212 to the proximalmost position, causing control wire 224 and control wire attachment 223 to be further moved proximally, and resulting in the closed configuration of first and second jaws 214, 216. A distance from stop 248 to the proximalmost position of spool 212 on handle 208 to may be a third distance that is less than the first distance between the proximalmost and distalmost positions of spool 212 on handle 208. A sum of the second distance and the third distance may be equal to the first distance.

Otherwise, if based on the impedance analysis, a determination is made that the portion of tissue is not likely an area of interest, the portion of tissue has not yet been pierced or severed from the body lumen. Therefore, rather than collect a negative sample, the operator can simply transition first and second jaws 214, 216 from the intermediate configuration back to the open configuration (e.g., perform a reverse of the second actuation by distally moving spool 212 to the distalmost position on handle 208), and receive a different portion of tissue for measurement and potential collection.

FIG. 7 depicts an example second sample collection device 700, hereinafter device 700 for brevity. Device 700 may be the same as device 200, except for an end effector assembly 704 that may replace end effector assembly 204 of device 200, and be connected to actuator assembly 202 via elongate member 206. End effector assembly 704 may include first and second jaws 714, 716. First and second jaws 714, 716 may be the same as first and second jaws 214, 216, except that no portion of first and second jaws 714, 716 is conductive or capable of forming electrodes 106. Rather, end effector assembly 704 includes first and second electrodes 706, 708 that are separate from (e.g., not integrated with a portion of) and are independently movable or actionable from first and second jaws 714, 716.

For example, when first and second jaws 714, 716 of device 700 are in an open configuration to receive a portion of tissue, initially first and second electrodes 706, 708 may be in an open configuration recessed within the body of first and second jaws 714, 716. For example, electrodes 706, 708 may be aligned with and received within jaws 714, 716. Then, as shown in FIG. 7, only first and second electrodes 706, 708 may be transitioned from the open configuration to a closed configuration (i.e., move radially inward toward a central longitudinal axis of device 700/end effector assembly 704) to cause first and second electrodes 706, 708 to provide constant contact and application of pressure to the portion of tissue to obtain an impedance measurement. A surface area of the contact and/or an amount of pressure applied to the portion of tissue as first and second electrodes 706, 708 are in the closed configuration may correspond to optimal value(s) for obtaining the impedance measurement.

First and second jaws 714, 716 may remain in the open configuration as the impedance of the portion of tissue is being measured and analyzed. The portion of tissue has not yet been pierced or severed from the body lumen given that first and second jaws 714, 716 remain in the open configuration. Therefore, if based on the impedance analysis, a determination is made that the portion of tissue is not likely an area of interest, rather than collect a negative sample, first and second electrodes 706, 708 may be transitioned from the closed configuration back to the open configuration, and receive a different portion of tissue for measurement and potential collection. Otherwise, if based on the impedance analysis, a determination is made that the portion of tissue is likely an area of interest and thus the portion of tissue should be collected as a sample, first and second jaws 714, 716 may be transitioned from the open configuration to the closed configuration to collect the sample. In the closed configuration of first and second jaws 714, 716, first and second electrodes 706, 708 may again be recessed within the body of first and second jaws 714, 716 to prevent interference with the collection of the sample.

In some examples, to facilitate the independent movement or actuation of first and second electrodes 706, 708 from first and second jaws 714, 716, a second control wire (not shown) separate from control wire 224 (e.g., a first control wire) may be connected to first and second electrodes 706, 708. Similar to control wire 224, the second control wire may extend proximally within elongate member 206 to handle 208 such that specific movements of spool 212 (e.g., utilizing stop 248) or movement of another (e.g., a second) actuator separate from spool 212 on handle 208 may cause first and second electrodes 706, 708 to transition between the open and closed configurations separate from the transition of first and second jaws 714, 716 between open and closed configurations.

As one example, when the portion of tissue has been received as both first and second electrodes 706, 708 and first and second jaws 714, 716 are in open configurations, spool 212 may be moved proximally from the distalmost position of spool 212 on handle 208 until spool 212 encounters stop 248, causing only the second control wire to move proximally, and resulting in the transition of first and second electrodes 706, 708 from the open to closed configuration. Then, dependent on the impedance analysis, first and second electrodes 706, 708 can be transitioned from the closed configuration back to the open configuration (e.g., by distally moving spool 212 to the distalmost position on handle 208), and receive a different portion of tissue for measurement and potential collection. Or if sample collection is desired, first and second jaws 714, 716 can be transitioned from the open configuration to the closed configuration to collect the portion of tissue. For example, force may be applied by the operator to overcome or otherwise release stop 248, and spool 212 may be further moved proximally to the proximalmost position, causing control wire 224 and control wire attachment 223 to be moved proximally, and resulting in the closed configuration of first and second jaws 714, 716. Alternatively, actuator assembly 202 may include a second actuator (e.g., spool, knob, lever, slider, etc.) for transitioning electrodes 706, 708 between open and closed configurations.

FIG. 8 depicts an example third sample collection device 800, hereinafter device 800 for brevity. Device 800 may be the same as device 200, except for an end effector assembly 804 that may replace end effector assembly 204 of device 200, and be connected to actuator assembly 202 via elongate member 206. End effector assembly 804 may include first and second jaws 814, 816. First and second jaws 814, 816 may be the same as first and second jaws 214, 216, except that no portion of first and second jaws 814, 816 is conductive or capable of forming electrodes 106. Rather, end effector assembly 804 includes an instrument 802 having first and second electrodes 806, 808 positioned at a distal end of instrument 802. One example of instrument 802 may include a bipolar needle or trocar. Instrument 802 may be longitudinally extendable and retractable from end effector assembly 804 (e.g., via central lumen 238 of clevis 218) when first and second jaws 814, 816 are in an open configuration. FIG. 8 shows instrument 802 in an extended configuration.

The extension and retraction of instrument 802 may be separately actionable from a transition of first and second jaws 814, 816 between open and closed configurations. For example, when the portion of tissue has been received as first and second jaws 814, 816 are in open configurations, instrument 802 may be moved distally (e.g., parallel to or coaxially with a central longitudinal axis of device 800 and/or end effector assembly 804) from the retracted to extended configuration. As instrument 802 is moved into the extended configuration, a distal end of instrument 802 may at least contact and/or partially pierce and penetrate the portion of tissue to obtain the impedance measurement. Then, based on the impedance analysis, if the portion of the tissue is not to be collected as a sample, instrument 802 may be moved proximally (e.g., parallel to or coaxially with a central longitudinal axis of device 800 and/or end effector assembly 804) from the extended configuration to at least a partially retracted configuration as first and second jaws 814, 816 may remain open to receive another portion of tissue. Alternatively, if the portion of the tissue is to be collected, spool 212 may be moved proximally relative to handle 208 to transition first and second jaws 814, 816 from the open configuration to the closed configuration. In some examples, instrument 802 may be transitioned from the extended configuration to the retracted configuration prior to transitioning first and second jaws 814, 816. In other examples, instrument 802 may remain in the extended configuration as first and second jaws 814, 816 are transitioned from the open configuration to the closed configuration. For example, instrument 802 may help to secure the tissue within tissue-receiving space 221.

In some configurations, a second control wire (not shown) separate from control wire 224 (e.g., a first control wire) may be connected to a distal end of instrument 802. Similar to control wire 224, the second control wire may extend proximally within elongate member 206 to handle 208 such that specific movements of spool 212 (e.g., utilizing stop 248) or movement of another actuator (e.g., a knob, spool, slider, lever, etc.) separate from spool 212 on handle 208 may cause instrument 802 to transition between retracted and extended configurations separate from the transition of first and second jaws 814, 816 between open and closed. The second control wire may, in some examples, house conductive wires therein that are attached to first and second electrodes 806, 808 and may exit handle 208 via port 250 to electrically couple first and second electrodes 806, 808 to computing device 108.

In other configurations, a distal end of instrument 802 may be inserted into handle 208 via port 250 and through elongate member 206 to end effector assembly 804 for receipt by central lumen 238 of clevis 218. A proximal end of instrument 802 may be manipulated by an operator to distally and proximally move the distal end of instrument 802 relative to end effector assembly 804 to transition instrument 802 between extended and retracted configurations. For example, the operator may interact with a handle separate from handle 208 to which the proximal end of instrument 802 is attached. Additionally, proximal end of instrument 802 may be electrically coupled to computing device 108.

As described in detail with reference to FIGS. 1-8, first configuration 300 of device 200, second configuration 400 of device 200, third configuration 500 of device 200, device 700, and device 800 are non-limiting and non-exhaustive examples of an sample collection device 104 that may be used in conjunction with medical device 102 to perform impedance-based biopsy.

FIG. 9 depicts an example process 900 for determining tissue impedance to guide a sample collection determination. In some examples, one or more steps or decisions of process 900 may be performed by processor(s) 112 of computing device 108 removably connectable to electrodes 106 of sample collection device 104. Illustrative examples included to provide context to the steps or decisions of process 900 describe sample collection device 104 as device 200. However, in other examples, sample collection device 104 may alternatively include device 700 or device 800.

Step 902 of process 900 may include to provide electrical signals to electrodes 106 of sample collection device 104. For example, during a biopsy procedure, medical device 102 may be inserted into and navigated through a body lumen of a patient to identify, using images captured by medical device 102, a target site within the body lumen from which tissue samples are to be collected. At least a distal portion of sample collection device 104, such as end effector assembly 204 of device 200, may be delivered to the target site via the medical device 102. First and second jaws 214, 216 may initially be in the closed configuration as end effector assembly 204 is delivered to the target site. Once delivered, first and second jaws 214, 216 may be transitioned from the closed configuration to the open configuration to receive a first portion of tissue within first and second jaws 214, 216. In some examples, the first portion of tissue received may be healthy tissue (e.g., non-suspect tissue) within the target site to enable an impedance baseline to be determined for the patient, as described in detail below with reference to step 902.

As first and second jaws 214, 216 have received the first portion of tissue, electrical signals may be generated by computing device 108. The generated electrical signals may include a current associated with one or more frequencies that may be provided from computing device 108 to electrodes 106 (e.g., forming at least a portion of body of first and second jaws 214, 216) via conductive wires 302, 304. The current is passed through the first portion of tissue and voltages resulting from an opposition of first portion of tissue to the current are received as a response signal for use in determining an impedance value of the first portion of the tissue for use as a baseline.

Current applied at varying frequencies may elicit different response signals from the first portion of tissue from which different tissue characteristics may be inferred or identified as part of the impedance analysis. For example, certain frequencies may enable differentiation of a type of tissue (e.g., fat, nerve, or muscle tissue), while other frequencies may enable differentiation of abnormal and healthy tissue and/or malignant and benign tumor tissue.

In some examples, step 902 may be repeated as two or more first portions of tissue (e.g., healthy portions of tissue) are received by first and second jaws 214, 216 to enable a more robust baseline impedance determination for the patient's healthy or normal tissue. In further examples, the electrical signals may be provided continuously through at least steps 904 and 906.

Step 904 of process 900 may include to determine a baseline impedance measurement based on first response signals received from electrodes 106 as electrodes 106 are contacting the first portion of tissue (e.g., healthy, non-suspect tissue). The baseline impedance measurement may be determined based on an amplitude and/or a phase of the voltage received as part of the first response signals. In examples where current is provided and applied at varying frequencies (e.g., where an impedance scan is performed), a baseline impedance measurement may be determined for each of the frequencies. Once a baseline impedance measurement is determined, the device may be moved or repositioned within the target site to receive a second portion of tissue that is believed to include potentially abnormal or suspect tissue (e.g., guided by images captured by medical device 102).

Step 906 of process 900 may include to determine an impedance measurement based on second response signals received from electrodes 106 as electrodes 106 are contacting the second portion of tissue (e.g., the potentially abnormal or suspect tissue). Similar to the baseline impedance measurement, the impedance measurement may be determined based on an amplitude and/or a phase of the voltage received as part of the second response signals. Additionally, in examples where current is provided and applied at varying frequencies to the suspect tissue (e.g., where an impedance scan is performed), an impedance measurement may be determined for each of the frequencies.

In some examples, as at least steps 904 and 906 are being performed, first and second jaws 214, 216 of first sample collection device 200 may be positioned in the intermediate configuration as shown in FIG. 6. Alternatively, when sample collection device 104 is device 700, electrodes 706, 708 may be positioned in the closed configuration as shown in FIG. 7. When sample collection device 104 is device 800, instrument 802 may be positioned in the extended configuration as shown in FIG. 8. Resultantly, constant contact surface area and pressure of values optimal for impedance may be applied to the first and second portions of tissue

Step 908 of process 900 may include to determine a difference between the baseline impedance measurement determined at step 904 and the impedance measurement determined at step 906. The difference may be compared to a predetermined threshold. In some examples, the predetermined threshold for the difference may be included as part of an impedance library stored in memory 110 and/or a remote data storage system (e.g., one of optional server side system(s) 118).

The difference meeting and/or exceeding the predetermined threshold may indicate that the second portion of tissue does have characteristics significantly different from the healthy first portion of tissue upon which the baseline impedance measurement was based, and thus the second portion of tissue likely does include suspect tissue that will be of interest to sample. The difference failing to meet and/or exceed the predetermined threshold may indicate that characteristics of the second portion of tissue are similar enough to the healthy first portion of tissue, that the second portion of tissue may also be healthy, and thus may likely result in a negative sample if collected.

In addition to determining the difference from the baseline impedance measurement, and particularly when the impedance scan is performed, the impedance measurement(s) determined at step 906 may further be analyzed to determine additional information associated with the suspect tissue, such as the type of tissue (e.g., whether the tissue is fat, muscle, nerve, etc.). In some examples, the predetermined threshold obtained from the impedance library to determine the difference may be specific to a type of tissue. For example, a different predetermined threshold may be associated with each of biliary duct tissue, gallbladder tissue, pancreatic tissue, etc., and/or may be further differentiated based upon whether the tissue is fat, muscle, nerve, etc. The impedance library may also store additional information useful for the impedance analysis, such as a range for average baseline impedance measurements, which again may be specific to the type of tissue.

Step 910 of process 900 may include to, based on the difference, generate and provide a user interface for display. The user interface may indicate whether to collect a sample of the second portion of the tissue. For example, if the difference meets and/or exceeds the predetermined threshold, the user interface may indicate to collect the second portion of tissue as the sample. Alternatively, if the difference fails to meet and/or exceed the predetermined threshold, the user interface may indicate to not collect the second portion of tissue as the sample. Example user interfaces are shown with reference to FIGS. 10A-B below.

In some examples, the user interface may display additional information determined as part of the process 900. A non-limiting and non-exhaustive listing of the additional information may include the baseline impedance measurement, the impedance measurement, the predetermined threshold, an indication of whether the baseline impedance measurement falls within an average range, and/or an indication of tissue type of the second portion of tissue.

Process 900 described above is provided merely as an example, and may include additional, fewer, different, or differently arranged steps than depicted in FIG. 9.

FIGS. 10A and 10B depict example first and second user interfaces 1000, 1010. First and second user interfaces 1000, 1010 may be generated and provided for display as part of step 910 of process 900 described above with reference to FIG. 9. In some examples, first and second user interfaces 1000, 1010 may be generated by computing device 108 and provided to one or more of display device(s) 116 that may be integrated with or separate from computing device 108.

FIG. 10A depicts first user interface 1000 that may be generated and displayed in response to computing device 108 determining that a portion of tissue likely includes suspect tissue based on an impedance analysis of the portion of tissue, and thus should be collected as a sample. As shown, first user interface 1000 may include a visual and/or textual indicator 1002 to clearly inform the operator of a recommendation to collect the sample, along with descriptive or explanatory text 1004.

FIG. 10B depicts second user interface 1010 that may be generated and displayed in response to computing device 108 determining that a portion of tissue does not likely include suspect tissue based on an impedance analysis of the portion of tissue, and thus should not be collected as a sample. Similar to first user interface 1000, second user interface 1010 may include a visual and/or textual indicator 1012 to clearly inform the operator of a recommendation to not collect the sample, along with descriptive or explanatory text 1014.

Although not shown in first and second user interfaces 1000, 1010, additional information determined as part of process 900 may be included. Example types of the additional information may include baseline impedance measurements, impedance measurements, differences between baseline impedance and impedance measurements, associated thresholds, and/or characteristics of the tissue (e.g., tissue type, malignant or benign).

First and second user interfaces 1000, 1010 described above are provided merely as examples, and may include additional, fewer, different, or differently arranged information and/or interactive elements than depicted in FIGS. 10A-B.

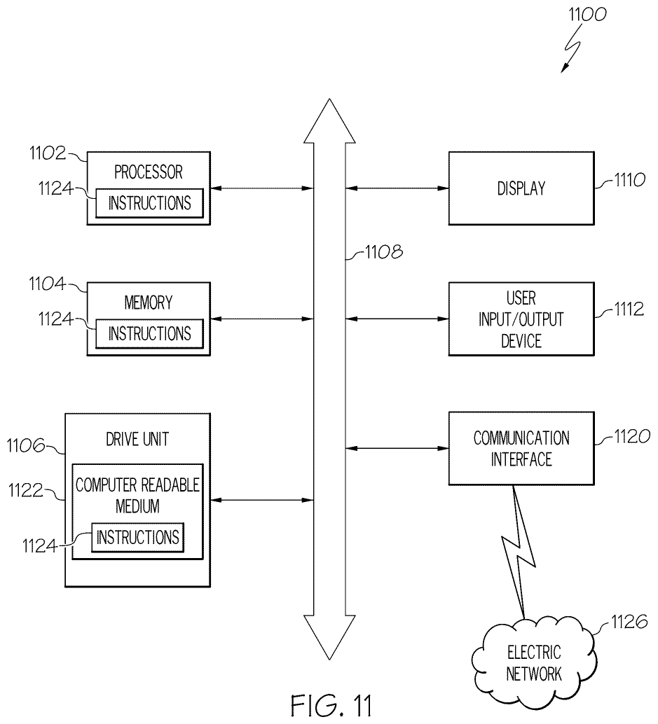

FIG. 11 depicts an example of a computer 1100. FIG. 11 is a simplified functional block diagram of computer 1100 that may be configured as a device for executing processes, steps, or operations depicted in, or described with respect to, FIGS. 9 and 10A-B and, according to exemplary embodiments of the present disclosure. For example, computer 1100 may be configured as one or more of medical device 102, computing device 108, display device(s) 116, optional server side system(s) 118, and/or another device or component according to exemplary embodiments of this disclosure. In various embodiments, any of the systems herein may be or include computer 1100 including, e.g., a data communication interface 1120 for packet data communication. Computer 1100 may communicate with one or more other computers, for example, using an electronic network 1126 (e.g., via data communication interface 1120). Electronic network 1126 may include a wired or wireless network, for example, similar to optional network 120 depicted in FIG. 1.

Computer 1100 also may include a central processing unit (“CPU”), in the form of one or more processors 1102, for executing program instructions 1124. Program instructions 1124 may include at least instructions for performing tissue impedance analysis (e.g., if computer 1100 is computing device 108).

Computer 1100 may include an internal communication bus 1108. Computer 1100 may also include a drive unit 1106 (such as read-only memory (ROM), hard disk drive (HDD), solid-state disk drive (SDD), etc.) that may store data on a computer readable medium 1122 (e.g., a non-transitory computer readable medium), although computer 1100 may receive programming and data via network communications. Computer 1100 may also have a memory 1104 (such as random-access memory (RAM)) storing instructions 1124 for executing techniques presented herein. It is noted, however, that in some aspects, instructions 1124 may be stored temporarily or permanently within other modules of computer 1100 (e.g., processor 1102 and/or computer readable medium 1122). Computer 1100 also may include user input and output devices 1112 and/or a display 1110 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
an end effector assembly at a distal end of the medical device, the end effector assembly comprising:
a pair of jaw members, each of the pair of jaw members including a jaw and a tang, the jaw being transitionable between an open configuration for receiving a portion of tissue and a closed configuration for collecting the portion of tissue as a sample; and
a pair of electrodes including a first electrode and a second electrode;
a first conductive wire attached to the first electrode; and
a second conductive wire attached to the second electrode,
wherein each of the first conductive wire and the second conductive wire extend to a proximal end of the medical device and are removably connectable to a computing device configured to generate and provide electrical signals to the pair of electrodes as the pair of electrodes are contacting the portion of the tissue, determine an impedance measurement of the portion of the tissue based on response signals received from the pair of electrodes, and generate and provide a user interface indicating whether to collect the portion of tissue as the sample based on the impedance measurement.

2. The medical device of claim 1, wherein each of the pair of electrodes is a portion of a body of the jaw of one of the pair of jaw members.

3. The medical device of claim 2, wherein the jaw of each of the pair of jaw members is further transitionable to an intermediate configuration for the impedance measurement, the intermediate configuration positioned between the open configuration and the closed configuration.

4. The medical device of claim 3, further comprising:
an actuator assembly at the proximal end of the medical device, wherein the actuator assembly includes a stop that maintains the jaw of each of the pair of jaw members in the intermediate configuration.

5. The medical device of claim 3, wherein, in the intermediate configuration, a contact surface area between the pair of electrodes and the portion of the tissue is maintained, and a pressure of a particular value is applied to the portion of the tissue.

6. The medical device of claim 1, wherein the pair of electrodes are separate from the pair of jaw members.

7. The medical device of claim 6, wherein the pair of electrodes are independently actionable from the pair of jaw members.

8. The medical device of claim 7, wherein, in response to an actuation of an actuator assembly, the pair of electrodes transition from an open configuration to a closed configuration for the impedance measurement as the pair of jaws remain in the open configuration.

9. The medical device of claim 8, wherein, in the closed configuration of the pair of electrodes, a contact surface area between the pair of electrodes and the portion of the tissue is maintained, and a pressure of a particular value is applied to the portion of the tissue.

10. The medical device of claim 1, further comprising:
an instrument having the pair of electrodes disposed on a distal end of the instrument.

11. The medical device of claim 10, wherein the instrument is transitionable between a retracted configuration and an extended configuration relative to the end effector assembly.

12. The medical device of claim 11, wherein the instrument is a needle, and in response to an actuation of an actuator assembly, the distal end of the instrument transitions to the extended configuration such that the distal end extends distally from the end effector assembly and at least partially into the portion of tissue for the impedance measurement as the pair of jaws remain in the open configuration.

13. The medical device of claim 1, further comprising:
a member connecting the end effector assembly to an actuator assembly at the proximal end of the medical device, wherein the first conductive wire and the second conductive wire extend within the member to the proximal end of the medical device.

14. The medical device of claim 13, further comprising:
a control wire attached to the actuator assembly and to the end effector assembly, and extending within the member, wherein the first conductive wire and the second conductive wire extend within the control wire to the proximal end of the medical device.

15. The medical device of claim 1, further comprising:
a member connecting the end effector assembly to an actuator assembly at the proximal end of the medical device, wherein the first conductive wire and the second conductive wire extend along an exterior of the member to the proximal end of the medical device.

16. A medical system comprising:
a sample collection device comprising:
a pair of jaw members, each of the pair of jaw members including a jaw and a tang, the jaw being transitionable between an open configuration for receiving a portion of tissue and a closed configuration for collecting the portion of tissue as a sample;
a pair of electrodes including a first electrode and a second electrode;
a first conductive wire attached to the first electrode; and
a second conductive wire attached to the second electrode; and
a computing device removably and electrically connected to the pair of electrodes via the first conductive wire and the second conductive wire, the computing device comprising:
at least one memory configured to store instructions; and
at least one processor configured to execute the instructions to perform operations comprising:
generating and providing electrical signals to the pair of electrodes;
determining an impedance measurement for the portion of tissue based on response signals received as the pair of electrodes are contacting the portion of tissue; and
based on the impedance measurement, generating and providing a user interface for display, the user interface indicating whether to collect the portion of tissue as a sample.

17. The medical system of claim 16, wherein the portion of tissue is a second portion of tissue associated with suspect tissue, the response signals are second response signals, and the operations further comprising:
determining a baseline impedance measurement based on first response signals received as the pair of electrodes are contacting a first portion of tissue associated with non-suspect tissue;
determining a difference between the baseline impedance measurement and the impedance measurement; and
comparing the difference to a predetermined threshold, wherein the user interface is generated further based on the comparing.

18. The medical system of claim 17, wherein generating the user interface comprises one of:
generating a first user interface to indicate to collect the second portion of tissue as the sample when the difference meets or exceeds the predetermined threshold; or
generating a second user interface to indicate not to collect the second portion of tissue as the sample when the difference fails to meet or exceed the predetermined threshold.

19. The medical system of claim 16, the operations further comprising:
determining a type of the tissue based on the response signals, wherein the user interface further displays the type of the tissue.

20. A method comprising:
performing a first actuation to move a control wire, connected to an end effector assembly, in a first direction to cause a conductive pair of jaws of the end effector assembly having received a portion of tissue therein to transition from an open configuration to an intermediate configuration, wherein a computing device to which the conductive pair of jaws are connected to provides electrical signals to, and determines an impedance measurement of the portion of tissue based on response signals received from the conductive pair of jaws that are contacting the portion of the tissue in the intermediate configuration; and
based on the impedance measurement, performing a second actuation to further move the control wire in the first direction to cause the conductive pair of jaws to transition from the intermediate configuration to a closed configuration to collect the portion of the tissue as a sample.
